(19) United States
(12) Patent Application Publication
(10) Pub. No.: US 2019/0192755 A1
(43) Pub. Date: Jun. 27, 2019

(54) POWER GENERATOR FOR USE IN LEFT VENTRICULAR ASSIST DEVICE (LVAD) AND TOTAL ARTIFICIAL HEART (TAH) AND RELATED METHODS

(71) Applicant: Claudio Filippone, College Park, MD (US)

(72) Inventor: Claudio Filippone, College Park, MD (US)

(21) Appl. No.: 16/325,183

(22) PCT Filed: Aug. 14, 2017
(86) PCT No.: PCT/US2017/046835

§ 371 Date: Feb. 12, 2019

Related U.S. Application Data
(60) Provisional application No. 62/374,799, filed on Aug. 13, 2016.
(60) Provisional application No. 62/374,832, filed on Aug. 13, 2016.

(51) Int. Cl. A61M 1/12; A61M 1/10; F04D 7/00; F04D 13/06; H02K 44/08; G21H 3/00

(52) U.S. Cl. CPC A61M 1/127; A61M 1/1015; A61M 1/1036; A61M 1/122; F04D 7/00; F04D 3/00; H02K 44/08; G21H 3/00; A61M 2205/368; A61M 2205/8287; F04D 13/06

(57) ABSTRACT

Various embodiments of a medical device for displacing a bodily fluid inside a patient's body and the related methods are disclosed. In one exemplary embodiment, the medical device may include a source heat exchanger containing a heat generating in source and being configured to transfer heat from the heat generating source to a working fluid. The medical device also includes a hollow shaft comprising a plurality of permanent magnets, an impeller shroud disposed inside the hollow shaft, where the impeller shroud defines an internal passageway through which the bodily fluid passes through. The medical device further includes an impeller disposed inside the internal passageway of the impeller shroud, where the impeller is magnetically coupled to the permanent magnets of the hollow shaft. The medical device includes an expander comprising a rotary component mechanically coupled to the hollow shaft, where the expander being driven by the working fluid flowing from the source heat exchanger to rotate the hollow shaft. Rotation of the hollow shaft generates a rotary magnetic field in the hollow shaft to cause the impeller to rotate and displace the bodily fluid flowing through the internal passageway.

This application is a U.S. National Stage Application of PCT International Application No. PCT/US2017/046835, filed Aug. 14, 2017, which claims the priority benefit to U.S. Provisional Application No. 62/374,799, filed Aug. 13, 2016, and U.S. Provisional Application No. 62/374,832, filed Aug. 13, 2016, the disclosures of which are hereby incorporated by reference in their entirety.

### FIELD OF INVENTION

The present invention relates generally to medical devices and related methods. More specifically, particular embodiments of the invention relate to implantable power generators for use with, for example, left ventricular assist devices (LVAD) and/or total artificial hearts (TAH).

### DESCRIPTION OF RELATED ART

An LVAD is a surgically implanted mechanical pump that is attached to the heart to assist pumping of blood from the left ventricle to the aorta. An LVAD includes a driveline extending from the pump to a controller positioned outside the patient's body and a power source connected to the controller to provide power to the pump. The power source usually includes batteries or live electricity. Depending on, for example, the patient condition and/or availability of a heart donor, an LVAD may be a temporary (e.g., weeks to several weeks) or permanent solution to failing heart. While an LVAD works with the heart to help it pump more blood with less work by the heart, a TAH is an artificial heat that completely replaces the failing heart.

SynCardia Systems, Inc. is a manufacturer of CardioWest™ Total Artificial Heart (TAH-t), which is an implantable artificial heart intended to keep hospitalized patients alive while they are waiting for a heart transplant. CardioWest™ TAH-t is a pulsating bi-ventricular device that is implanted into the chest to replace the patient's left and right ventricles (the bottom half of the heart). The device is sewn to the patient's remaining atria (the top half of the heart). Hospitalized patients are connected by tubes from the heart through their chest wall to a large power-generating console, which operates and monitors the device.

AbioCor™ is an implantable, self-contained total artificial heart produced by ABIOMED. AbioCor™ is formed by an implanted pump, an internal rechargeable battery capable of supporting operation for 20 minutes, continuously charged by an external power source, and an electronic package implanted in the patient's abdominal area. Power to recharge the implanted battery is transferred via transcutaneous energy transmission (TET) system. External battery packs can power AbioCor™ for 4 hours. AbioCor™ was discontinued in 2007.

CARMAT is developing an implantable artificial heart equipped with electrical power supply and remote diagnosis systems. The artificial heart consists of two, right and left, ventricular cavities containing two volume spaces each separated by a flexible bio-membrane, one for blood and one for a working fluid. Through hydraulic action via two motorized pump sets, the working fluid displaces the bio-membrane, thus reproducing the movement of the ventricular wall of the human heart. An integrated electronic device regulates how the artificial heart operates according to patients' needs and using information given by sensors and processed by a microprocessor.

Both LVADs and TAHs, including the particular devices mentioned above, require a mechanical or electro-mechanical pump that requires a sustained high-density power source external to the patient's body (e.g., external batteries and power supplied networked with the power grid or other types of electric generators).

Thus, there exists a need for an improved power generator that can provide a sustained, high-density power source with long-term energy storage capacity.

### SUMMARY

Therefore, various exemplary embodiments of the invention may provide an improved power generator that overcomes one or more shortcomings and problems of existing LVADs and TAHs. It should be understood that, while the power generator of the present disclosure is described in connection with a LVAD and TAH, the power generator may be applied in many other application that may require power sources with high energy density and long-term energy storage capacity.

For example, robotic applications require electrical power normally supplied by cables or tethers connected to stationary or mobile electric power supplies. For robotics applications requiring high power density and low weight, in addition to dimensional constraints as required, for example, by unmanned vehicles, aerial and submergible drones, electric power from portable solar panels or combustion engines can become unpractical or impossible. For example, man and unmanned submergible, non-nuclear electric robots cannot rely on solar or combustion engines. The power generator of the present disclosure may provide an autonomous rotary magnetic drive configured to convert thermal energy from nuclear decay heat can satisfy requirements for robotic applications.

In certain exemplary aspects, the rotary magnetic drive of the present disclosure can be totally implanted inside a patient's body and configured to convert decay heat energy into a rotary magnetic field executing the functions currently executed by the electro-magnetic or permanent magnet motors equipping FDA approved LVADs and TAHs pumping systems. The rotary magnetic drive can also be configured to convert decay heat thermal energy into conditioned electricity, thus replacing the battery and power supply system normally supplying electric power to LVADs and TAH. The rotary magnetic drive of the present disclosure can be scaled and configured to be totally implantable with no need for percutaneous tethers or drivelines to supply electric power to LVADs and TAHs.

When the rotary magnetic drive is configured to support medical applications, it represents an implantable energy source based on safely encased alpha-emitting isotopes that release thermal energy as they undergo natural nuclear-decay. In one embodiment, the thermal energy released by the alpha-emitting isotopes is converted into motive power or electricity by a miniaturized thermodynamic engine configured to exchange thermal energy with the environment through the body's natural heat transfer mechanisms.

Alpha-emitting isotopes are often referred to as soft radiation represented by Helium particles ejected by isotopes that undergo natural alpha-decay, and can easily be stopped by thin materials such as a sheet of paper, thus effectively shielding the alpha-emitting isotopes. For these applications, the alpha-emitting isotopes represent the power source of the rotary magnetic drive, and can be produced and manufactured in the form of compact shielded cartridges for simplified installation, removal or replacement at intervals dictated by the LVADs and TAH uninterrupted power generation rate and time duration requirement. The amount of alpha-emitting isotope required to power LVADs and TAHs and the power rating corresponding to the thermal energy released by the alpha-particles depends on the decay rate of the isotopes selected and the isotopes half-life. In other words, the total thermal power produced by the power source is directly proportional to the rate of alpha particles generation, while the duration at which the total thermal power can be produced depends on the isotopes half-life.

There are various alpha emitting isotopes that can provide thermal energy and time duration with specifications that satisfy LVADs and TAH application requirements. Most of the available alpha-emitting sources represent adequate power rating and half-life for LVADs applications. However, several of the available alpha-emitting isotopes are not pure alpha-emitters, as the primary alpha-emission may be emitted all together with secondary gamma-ray emissions. In most cases, the gamma-ray emission occurs at a very low rate, relative to the alpha emission, and with energy ranges that can be stopped by adequately designed shields. Shielding requirements for the power source become proportionally more restrictive depending on the type of gamma-rays emitted and their emission frequency. For LVADs and TAH applications, shielding of the power source is necessary to absorb gamma-radiation rather than alpha-particles, and to ensure patients and the public in their surrounding environments are not exposed to harmful radiation.

On average, LVADs require approximately 3-10 Watt-electric to electro-magnetically drive the blood pumping LVADs magnetic rotors. This power rating may increase when the LVADs or TAHs are configured to execute blood pumping by positive displacement or pneumatic mechanisms. For configurations involving rotary equipment as part of the blood pumping mechanisms (e.g., impeller rotors), the actual thermal power source rating increases accounting for electric-to-mechanical conversion inefficiencies.

In one embodiment of this invention, when the source energy is converted into a rotary magnetic field, thermal energy from the decaying isotopes is directly converted into motive (pumping) power by magnetic coupling with the permanent magnets comprised by the rotary blood pumping impeller. A certain portion of the thermal energy that is not converted into electricity or mechanical power is rejected to the environment by thermally coupling the rotary magnetic drive low temperature heat exchanger to the patient body to execute natural/passive or active convective, conductive and radiative heat transfer mechanisms.

Alpha-emitting isotopes safely encased within a heat transfer and shielding reinforced housing can produce thermal energy. This thermal energy is then converted into forms that can support robotic actuation and management, as well as LVADs and TAHs devices whose pumping functions are executed by magnetic rotary impellers or linear and positive displacement actuators. The amount of thermal energy produced is proportional to the isotope's natural decay-rate, while the duration at which thermal energy is released is proportional to the isotope's half-life. One of the candidate alpha-emitting isotopes include Plutonium-238 with a half-life of approximately 87 years. The main Pu238 nuclear decay mode is the alpha emission followed by a very low-energy secondary gamma ray emission. Therefore, among various isotopes, Plutonium-238 shielded with reasonably compact radiation shields can be utilized as a thermal source for the rotary magnetic drive of the present disclosure.

One exemplary aspect of the present disclosure may provide a magnetic drive electric and torque generator configured to convert thermal energy from a heat source into mechanical energy to drive a rotary magnetic field and further convert the rotary magnetic field in mechanical torque through magnetic coupling with a mechanical rotary system and into electric energy through magnetic coupling with stationary electro-magnetic coils. Rotary magnetic drive can be configured to support various applications, such as, for example, to drive the impeller of a pump, the propeller of a submergible vehicle, fans, and other generic actuators supporting robotic propulsion and actuation. Size and power rating of the rotary magnetic drive generator of the present disclosure can be scalable enabling totally implantable applications as required by blood pumping devices represented, for example, by LVADs and TAHs.

Further, the rotary magnetic drive generator can be configured as an implantable, autonomous, pumping power-generator to replace external or implantable rechargeable batteries and electro-magnetic motors normally equipping LVADs and TAHs. In one exemplary configuration, the rotary magnetic drive may convert thermal energy generated by a heat source, such as nuclear isotopes undergoing nuclear decay, into mechanical energy that drives a rotary magnetic field that can be coupled to various components to generate torque, propulsion, or electricity. In one another exemplary configuration, the rotary magnetic drive can be configured to drive blood pumping magnetic impellers in LVADs and TAHs to eliminate the need to rely on batteries with limited capacity and access to electric power supplies outside of the patient's body. As the rotary magnetic drive can be configured to produce mechanical energy at scalable power ratings, it can also be utilized to support electric generation for robotic applications.

Another exemplary aspect of the present disclosure may provide a power generator capable of supplying variable power ratings for a prolonged period of time based on generic thermal sources, including thermal sources represented by nuclear decaying isotopes. The power generator of the present disclosure may satisfy one or more of the following conditions: i) light weight and fully contained within dimensions and weight requirements characterizing various robotic and specialized applications, including LVADs and TAHs applications; ii) safe, as alpha radiation and low-energy secondary emission gamma rays are shielded by high density materials and by additional means represented by the shape of the materials forming the thermal-hydraulic heat exchanger, utilized to transfer thermal energy from the decaying isotopes to the working fluid, and the working fluid itself as its composition can comprise gamma-ray shielding materials; iii) does not require refueling or recharging of the power source for extended amounts of time (months to decades, depending on the half-life of the isotopes selected0; iv) contains rotary components that are not in contact with one another, thus ensuring frictionless “no wear and tear” operations; v) compactness, modular for integration with the equipment supporting robotic applications, and implantable for medical applications; vi) self-sustained automatic operations, no need for monitoring of functions; vii) for medical application it can be interfaced directly with FDA approved LVADs and TAHs via magnetic coupling; viii) provides extra shielding capabilities by means of routing the radiation-attenuating working fluid configured to circulate within heat exchangers transferring thermal energy from the decaying isotopes to the working fluid, while forming a “fluid wall thickness” that effectively attenuates alpha, beta and gamma radiation; ix) comprises a thermal power source whose decaying isotopes are fully encapsulated, sealed and inaccessible; x) provides power sources configurations wherein the decaying isotopes are manufactured in sealed cartridges formed by materials that satisfy thermal heat transfer and shielding capabilities; xi) can withstand hostile operations without releasing volatiles forms of the isotopes utilized for the generation of thermal energy, even under design basis and beyond design basis accident scenarios, including maliciously breaching of the fuel cartridge; and xii) complies with regulatory requirements for ionizing radiation.

To attain the advantages and in accordance with the purpose of the invention, as embodied and broadly described herein, one aspect of the invention may provide a medical device for displacing a bodily fluid inside a patient's body. In one exemplary embodiment, the medical device may include a source heat exchanger containing a heat generating source and being configured to transfer heat from the heat generating source to a working fluid. The medical device also includes a hollow shaft comprising a plurality of permanent magnets, an impeller shroud disposed inside the hollow shaft, where the impeller shroud defines an internal passageway through which the bodily fluid passes through. The medical device further includes an impeller disposed inside the internal passageway of the impeller shroud, where the impeller is magnetically coupled to the permanent magnets of the hollow shaft. The medical device includes an expander comprising a rotary component mechanically coupled to the hollow shaft, where the expander being driven by the working fluid flowing from the source heat exchanger to rotate the hollow shaft. Rotation of the hollow shaft generates a rotary magnetic field in the hollow shaft to cause the impeller to rotate and displace the bodily fluid flowing through the internal passageway.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several exemplary embodiments of the invention and together with the description, serve to explain the principles of the invention.

FIG. 1 is a schematic view of a power generator, according to an exemplary embodiment of the present disclosure, illustrating the basic thermal-hydraulic connections among various components forming a closed-loop thermodynamic cycle.

FIG. 2 is a perspective, partial cut-away view of a power conversion assembly, according to one exemplary embodiment of the present disclosure.

FIG. 3 is a cross-sectional view of the power conversion assembly shown in FIG. 2, shown with an expander integrally formed with a hollow shaft.

FIG. 4 is a schematic view of a power generator, according to another exemplary embodiment of the present disclosure.

FIG. 5 is a schematic diagram of a power generator, according to another exemplary embodiment.

FIG. 6 is a schematic view of a power generator, according to another exemplary embodiment.

FIG. 7 is a schematic view of a power generator, according to another exemplary embodiment.

FIG. 8 is a schematic diagram of a power generator, according to another exemplary embodiment.

FIG. 9 is a schematic diagram of a power generator, according to another exemplary embodiment.

FIG. 10 is a schematic diagram of a power generator, according to another exemplary embodiment.

FIG. 11 is a perspective view of the power generator described by FIGS. 1-3, according to one exemplary embodiment.

FIG. 12 is a perspective cross-sectional view of the power generator shown in FIG. 11, illustrating various internal components.

FIG. 13 is an exploded view of the power generator shown in FIGS. 11 and 12, illustrating various parts of the power generator.

FIG. 14 is a perspective cross-sectional view of a recuperator heat exchanger of the power generator shown in FIGS. 11-13.

FIG. 15 is a partially exploded perspective view of the power generator of FIG. 11.

FIG. 16 is a perspective view of the recuperator heat exchanger of the power generator of FIG. 11.

FIG. 17 is a perspective view of power generator 100 of FIG. 11, illustrating a different angle of the extended recuperator.

FIG. 18 is a perspective view of the power generator shown in FIGS. 6-10.

FIG. 19 is a perspective cross-sectional view of the power generator shown in FIG. 18.

FIG. 20 is a perspective view of the power generator coupled to an extended heat exchanger, according to an exemplary embodiment of the invention.

FIG. 21 is a transparent perspective view of the power generator and the extended heat exchanger of FIG. 20, illustrating the approximate positions of the power generator and the extended heat exchanger when implanted in a patient body.

FIG. 22 is a perspective view of a power generator coupled to an extended heat exchanger, according to another exemplary embodiment.

FIG. 23 is a functional schematic diagram of the power generator and extended heat exchanger of FIG. 22, illustrating the flow patterns of the working fluid in and out of the power generator 100.

FIG. 24 is a transparent perspective view of the power generator and the extended heat exchanger of FIG. 22, illustrating the approximate positions of the power generator and the extended heat exchanger when implanted in a patient body.

FIG. 25 is a perspective view of a power generator coupled to an extended heat exchanger, according to another exemplary embodiment.

FIG. 26 is a transparent perspective view of the power generator and the extended heat exchanger of FIG. 25, illustrating the approximate positions of the power generator and the extended heat exchanger when implanted in a patient body.

### DESCRIPTION OF THE EMBODIMENTS

Reference will now be made in detail to the exemplary embodiments consistent with the present invention, examples of which are illustrated in the accompanying drawings. Wherever possible, the same reference numbers will be used throughout the drawings to refer to the same or like parts.

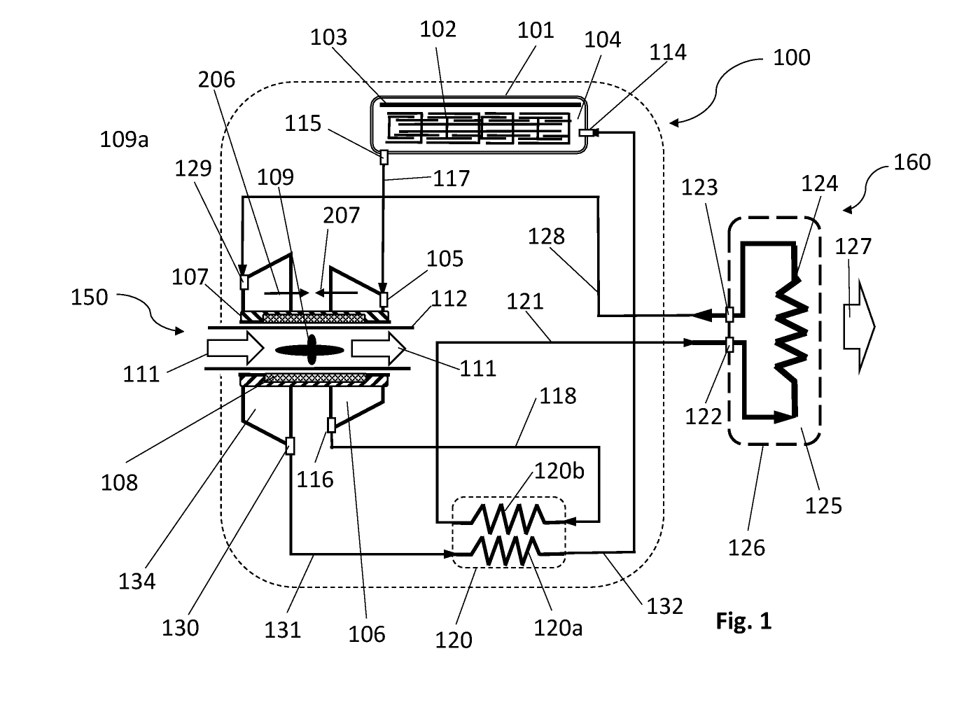

FIG. 1 schematically illustrates various components constituting a power generator 100 incorporating a power conversion assembly 150 for use in, for example, a LVAD or TAH, according to one exemplary embodiment of the present disclosure. While the present invention will be described in connection with a particular type of a LVAD or TAH, various aspects of the present disclosure may be used with any other types of LVADs and/or TAHs. Moreover, certain aspects of the inventions may be applied to, or used in connection with, any other device or machine that may need an uninterrupted, long-term power supply, such as, for example, robotics, propulsion devices, and actuators, some of which will be described throughout the disclosure.

As shown in FIG. 1, various components of power generator 100 are thermal-hydraulically interconnected to operate in a closed-loop Rankine thermodynamic cycle with a working fluid 104. Working fluid 104 may comprise any fluid that exhibits adequate thermal-physical properties to execute thermodynamic power cycles. In some exemplary embodiments, working fluid 104 may be an organic fluid. Working fluid 104 may also contain high-density materials, such as, for example, lead- or tungsten-based material, to function as radiation shielding.

Power generator 100 may include a housing 101 containing a source heat exchanger 102, a power conversion assembly 150, a recuperator heat exchanger 120, and a heat sink interface 160 for thermally communicating with an ultimate heat sink 127.

Housing 101 may be a sealed containment enclosing source heat exchanger 102 therein and having an inlet 114 and an outlet 115. Source heat exchanger 102 may include a heat generating source and one or more heat transfer channels and surfaces coupled to the heat generating source to transfer heat from the heat generating source to working fluid 104. As will be described in more detail later, in some exemplary embodiments, the heat generating source may include a nuclear material that releases decay heat. For example, the nuclear material that releases decay heat may include nuclear isotopes emitting alpha particles, such as, for example, Pu²³⁸. In alternative embodiments, source heat exchanger 102 may include or coupled to other types of thermal energy source, such as, for example, combustion products, solar cells, and geothermal source, depending on the type of application for which the power generator of the present disclosure may be used.

Housing 101 may be configured to thermally insulate source heat exchanger 102 from the environment surrounding housing 101. Housing 101 may also include a radiation shield 103 that substantially surrounds source heat exchanger 102 to protect the surrounding from radiation emitted by the nuclear material. In some exemplary embodiments, housing 101 may be sufficiently large to contain an inventory of working fluid 104. The structural configuration of housing 101 and source heat exchanger 102 will be described in detail later.

Power conversion assembly 150 may include a hollow shaft 107, an expander 106 having single- or multi-stage power turbine rotors mechanically coupled to hollow shaft 107, a pump 134 having one- or multi-stage turbine rotors mechanically coupled to hollow shaft 107.

As will be described in more detail later, the turbine rotors of pump 134 may be mechanically coupled to a proximal portion of hollow shaft 107, and the turbine rotors of expander 106 may be mechanically coupled to a distal portion of hollow shaft 107. To minimize axial shifts of hollow shaft 107 due to the thrust effects of working fluid 104 when compressed by pump 134 and expanded in expander 106, the turbine rotors of pump 134 and the turbine rotors of expander 106 can be arranged in a way that the directions of pump thrust 206 and expander thrust 207 are opposed against one another to minimize or nullify the thrust effects.

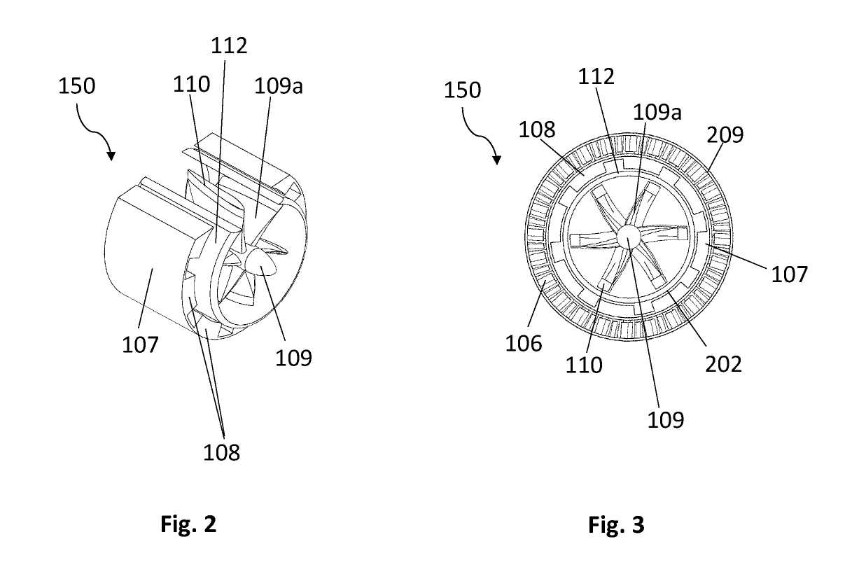

FIG. 2 is a perspective view of an exemplary power conversion assembly 150 with its top portion and expander 106 (see FIG. 3) removed to better illustrate the internal components therein. FIG. 3 is a cross-sectional view of power conversion assembly 150, illustrating its various rotary and stationary components. As shown in FIG. 3, expander 106 may be fixed to or integrally formed with hollow shaft 107. In this embodiment, expander 106 includes an expander casing 209 concentrically disposed over hollow shaft 107 and a plurality of fins or blades extending from one or both of an interior surface of expander casing 209 and an exterior surface of hollow shaft 107. If expander 106 is integrally formed with hollow shaft 107, expander casing 209 may represent an outer wall of hollow shaft 107, and the plurality of fins or blades may extend from the interior surface of expander casing 209.

As shown in FIGS. 2 and 3, power conversion assembly 150 may also include an impeller shroud 112 disposed inside hollow shaft 107 and an impeller 109 disposed inside impeller shroud 112. Hollow shaft 107 and impeller shroud 112 are concentrically arranged with respect to the rotational axis of impeller 109. Impeller shroud 112 defines an internal passageway through which a fluid to be pumped 111 (i.e., blood of a patent in case of an LVAD or TAH) can pass through.

Impeller shroud 112 may be stationary, and impeller 109 may be magnetically suspended inside impeller shroud 112. For example, on the interior wall or surface of hollow shaft 107, a plurality of permanent magnets 108 are radially disposed (e.g., embedded with or fixed to hollow shaft 107) about the rotating axis of impeller 109 to magnetically couple impeller 109 to permanent magnets 108. When hollow shaft 107 rotates as a result of an expansion by a working fluid 104 inside expander 106, permanent magnets 108 generate rotary magnetic fields that magnetically couple impeller 109 and exerts rotational forces on impeller 109 (e.g., similar to that generated by coils with a stator and/or rotor of an electrical motor), thereby exerting rotational forces on impeller 109.

In some exemplary embodiments, magnetic coupling between permanent magnets 108 and impeller 109 can be enhanced by magnetizing impeller blades 109a. Alternatively, magnetic coupling between permanent magnets 108 and impeller 109 can be enhanced by attaching permanent magnets to tips 110 of blades 109a, as shown in FIGS. 2 and 3. As a result, the rotary magnetic fields generated by permanent magnets 108 is converted into mechanical pumping power exerted onto the fluid 111 (e.g., blood) passing through the internal passageway defined by shroud 112.

In addition or as an alternative to the magnetic coupling between permanent magnets 108 and impeller 109, impeller 109 may be mechanically supported via bearings structurally coupled to impeller shroud 112 without significantly obstructing the flow of fluid 111 in the internal passageway defined by impeller shroud 112.

In some exemplary embodiments, hollow shaft 107 may be configured to float over impeller shroud 112 via working fluid 104. For example, hollow shaft 107 and impeller shroud 112 may be configured in a way that working fluid 104 can form hydrodynamic films in an annular gap 202 between the inner surface of hollow shaft 107 and the outer surface of impeller shroud 112, as shown in FIG. 2. Accordingly, working fluid 104 provides a low-friction, non-contact interface between impeller shroud 112 and hollow shaft 107 without requiring any additional a lubricant or friction-reducing material.

In some exemplary embodiments, to ensure concentricity of impeller 109 when fluid 111 passing through impeller shroud 112 exerts loading forces on impeller 109, the tips 110 of blades 109a of impeller 109 may be shaped to cause fluid 111 to form hydrodynamic films in the gap between the tips 110 of blades 109a and the inner surface of impeller shroud 112. The hydrodynamic films may allow impeller 109 to remain in a concentric position, thus creating low-friction, hydrostatic and hydrodynamic bearings.

The internal passageway defined by impeller shroud 112 is isolated from the closed-loop circuit of working fluid 104 to prevent mixing of working fluid 104 and fluid 111 passing through the internal passageway of impeller shroud 112. In addition, impeller shroud 112 may be made of a thermal insulating material to inhibit heat transfer between working fluid 104 and fluid 111.

In an alternative embodiment, where heat transfer between working fluid 104 and fluid 111 is desired, impeller should 112 may be made of a material exhibiting high thermal conductivity to enhance heat transfer between working fluid 104 and fluid 111.

As mentioned above, power generator 100 consistent with the present disclosure may be used to support various applications. For example, power generator 100 of the present disclosure may be used to actuate various types of actuators (e.g., linear or rotary actuators), and fluid 111 in communication with the internal passageway of impeller shroud 112 may be hydraulic oil used to pressurize the actuators. When power generator 100 of the present disclosure is applied to support propulsion, impeller 109 can be retrofitted with a propeller for submerged applications, where fluid 111 in the internal passageway of impeller shroud 112 can be a liquid (e.g., water or liquid metal) or gas (e.g., air).

With reference to FIG. 1, the thermodynamic cycle of power generator 100 will be explained. Working fluid 104 is pressurized by a single- or multi-stage turbine rotors of pump 134. Pressurized working fluid 104 exits an outlet 130 of pump 134 and enters a low-temperature portion 120a of recuperator heat exchanger 120 via a high-temperature channel 131. Working fluid 104 exiting an outlet 116 of expander 106 enters a high-temperature portion 120b of recuperator heat exchanger 120 via a low-temperature channel 118. Low-temperature portion 120a and high-temperature portion 120b of recuperator heat exchanger 120 are configured to exchange heat with one another. Accordingly, as pressurized working fluid 104 from pump 134 passes through recuperator heat exchanger 120, working fluid 104 is pre-heated to increase its energy content by heat transfer from working fluid 104 flowing from expander 106 and through high-temperature portion 120b.

Pressurized and pre-heated working fluid 104 exits recuperator heat exchanger 120, passes through a high-pressure channel 132, and enters a housing 101 via an inlet 114. Inside housing 101, working fluid 104 flows through source heat exchanger 102 and is further heated to increase its energy content by heat transfer from the heat generating source (e.g., decay heat from alpha-emitting nuclear isotopes).

With increased energy content, working fluid 104 exits source heat exchanger 102 of housing 101 and flows into expander 106 via one or more high-temperature channels 117. In one exemplary embodiment, high-temperature channel 117 may be configured to support the functions of recuperator heat exchanger 120. In another exemplary configuration, high-temperature channel 117 may be configured to thermally insulate working fluid 104 from the environment surrounding high-temperature channel 117.

As working fluid 104 enters expander 106 via an inlet 105, it expands and rotates the turbine rotors of expander 106 coupled to hollow shaft 107 (see also FIG. 3), thereby converting the thermal energy of working fluid 104 into mechanical energy in the form of torque applied to hollow shaft 107.

Torque applied to hollow shaft 107, in turn, rotates the turbine rotors of pump 134 to pressurize working fluid 104. Further, as described above, rotating hollow shaft 107 creates rotary magnetic fields by permanent magnets 108 mechanically coupled to or embedded in hollow shaft 107. Since permanent magnets 108 are magnetically coupled to impeller 109, the rotary magnetic fields generated by rotating hollow shaft 107 exert rotational forces on impeller 109.

When working fluid 104 is discharged from outlet 116 of expander 106 to low-temperature channel 118, its energy content is relatively low (e.g., proportional to the efficiency of expander 106). Low-temperature channel 118 may be configured to insulate working fluid 104 from the surrounding. In one exemplary embodiment, low-temperature channel 118 may constitute a portion of recuperator heat exchanger 120.

After exchanging thermal energy in recuperator heat exchanger 120, working fluid 104 flows into heat sink interface 160 via a channel 121 and an interface inlet 122 for thermally communicating with ultimate heat sink 127. When power generator 100 of the present disclosure is used in a LVAD or TAH, heat sink interface 160 may be implanted inside a patient's body along with power generator 100, where heat sink interface 160 exchanges heat energy with a patient's body portion (e.g., tissues, bones, body fluids, skin surface) via various heat transfer mechanisms (e.g., conductive, convective, and radiative) to reject thermal energy to ultimate heat sink 127 (e.g. air surrounding the patient).

In an exemplary embodiment, as shown in FIG. 1, heat sink interface 160 may include an extended heat exchanger 124 having heat transfer surfaces that, depending on the type of LVAD or TAH (or other application), allow heat transfer between working fluid 104 and a first thermal interface 125. First thermal interface 125 may be a sealed tank enclosing extended heat exchanger 124 with a cooling fluid. For example, first thermal interface 125 may be a pool of bodily fluid (e.g., urine inside a patient's bladder), and extended heat exchanger 124 can be submerged in the pool of bodily fluid. In an alternative embodiment, extended heat exchanger 124 may include a solid thermal interface 125 with a high thermal conductivity, such as a metallic element implanted in a patient's body.

Heat sink interface 160 may further include a second thermal interface 126 for allowing further heat transfer between working fluid 104 and ultimate heat sink 127. For example, second thermal interface 126 may include a pass-through mesh thermally coupled to ultimate heat sink 127. In another exemplary embodiment, second thermal interface 126 may be configured to enable a fluid of ultimate heat sink 127 to mix with the fluid of first interface 125.

The configuration of extended heat exchanger 124 in relation to first thermal interface 125 and second thermal interface 126 may vary significantly depending on the type of LVAD or TAH (or other applications) and the patient conditions. For example, for non-medical applications, such as, for example, propulsion, actuation, or robotics, extended heat exchanger 124 can be configured to transfer thermal energy from working fluid 104 directly to the ultimate heat sink 127 via finned radiators thermally coupling working fluid 104 with the air and/or water environments.

After being cooled down by extended heat exchanger 124 and with its temperature at its lowest value with respect to the thermodynamic Rankine cycle, working fluid 104 exits extended heat exchanger 124 via an outlet 123. Working fluid 104 then flows into an inlet of pump 134 via a cold channel 128, thus resetting the thermodynamic cycle of working fluid 104. In one exemplary embodiment, cold channel 128 can be thermally coupled to extended heat exchanger 124 to further extend its heat transfer surfaces and further increase condensing effectiveness of working fluid 104 prior to entering pump 134.

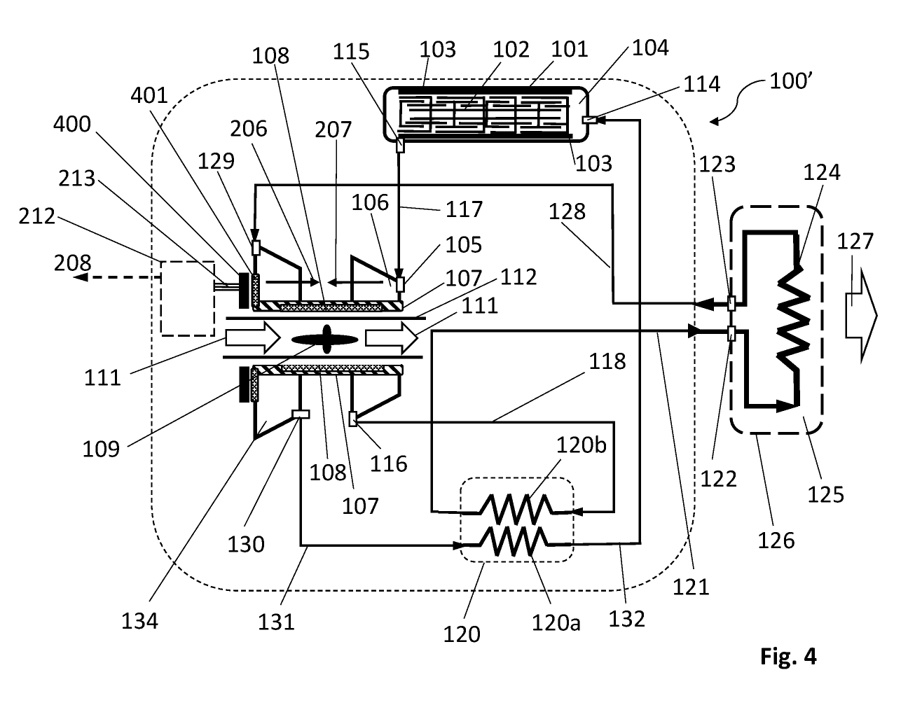

FIG. 4 is a schematic view of a power generator 100′, according to another exemplary embodiment of the present disclosure. One of the main differences between power generator 100′ shown in FIG. 4 and power generator 100 described above with reference to FIG. 1 is that power generator 100′ includes radial magnets 401 and an electromagnetic stator 400 to produce electricity and mechanical torque. Radial magnets 401 are mechanically coupled to hollow shaft 107, and a variable magnetic field is generated by radial magnets 401 when hollow shaft 107 rotates. Electromagnetic stator 400 comprising integrated electric coils is configured to convert the variable magnetic field into electricity conditioned by a controller 212.

The generated electricity in the form of AC or DC is then transmitted through integrated leads 213 to controller 212. Controller 212 is configured to condition the AC or DC electricity produced by electromagnetic stator 400 to supply power to various instrumentation and/or processing systems, such as, for example, sensors and data acquisition and processing systems that may provide information indicative of the performance of power generator 100. Controller 212 may also be configured to transmit the information wirelessly to an external device via an antenna 208.

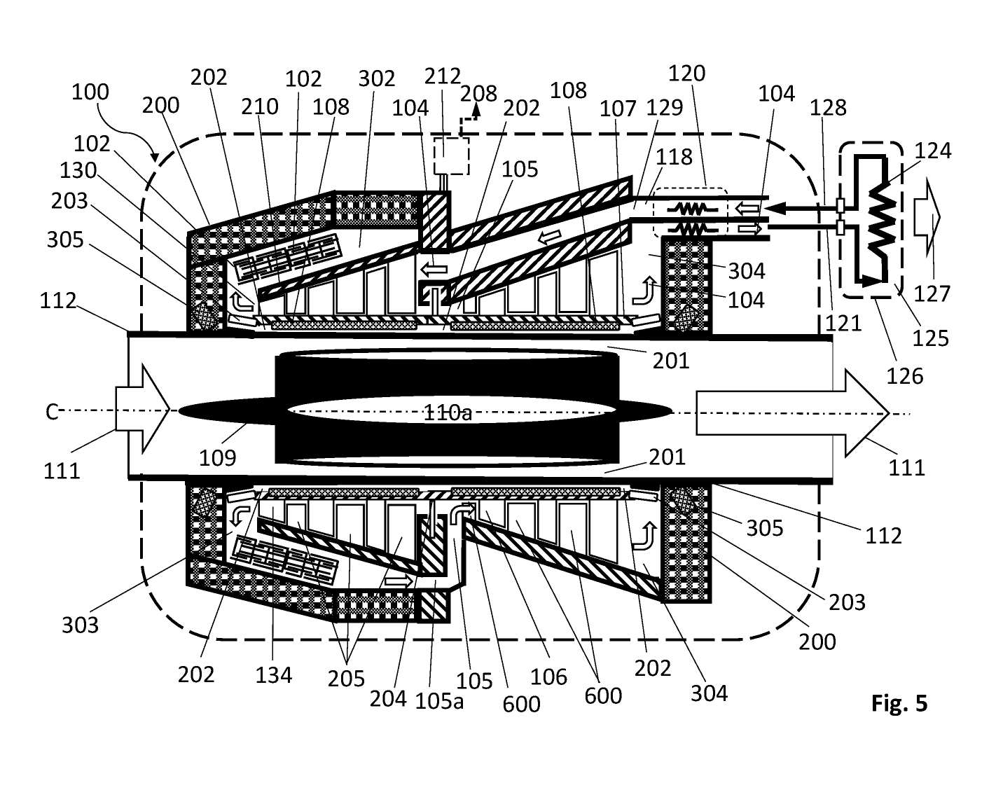

FIG. 5 is a schematic, functional diagram of power generator 100 with enhanced structural details, according to various exemplary embodiments of the present disclosure. In this embodiment, source heat exchanger 102 is integrally formed with power conversion assembly 150, and power generator 100 includes a shield 200 substantially surrounding source heat exchanger 102. Shield 200 may be provided in addition to or in alternative to radiation shield 103 shown in FIGS. 1 and 4.

As shown in FIG. 5, source heat exchanger 102 may be formed of a conically- or cylindrically-shaped annular heat exchanger and configured to contain a heat generating source (e.g., alpha-emitting isotopes). Pump 134 may include a pump shroud 210 to which a plurality of pump stators 205 are attached. Source heat exchanger 102 may substantially surround pump shroud 210.

Starting from extended heat exchanger 124, condensed working fluid 104 flows through cold channel 128 and enters recuperator heat exchanger 120. Low-temperature portion 120a and high-temperature portion 120b of recuperator heat exchanger 120 may be formed of two concentric annular channels with a wall separating the annular channels serving as the heat transfer surfaces. Working fluid 104 then enters inlet 129 of pump 134 to be pressurized through multi-stage turbine rotors 134 and pump stators 205.

At outlet 130 of pump 134, working fluid 104 is pressurized and enters source heat exchanger 102 to increase its energy content via thermal exchange with the heat generating source contained in source heat exchanger 102. After flowing circumferentially and axially through source heat exchanger 102, working fluid 105 flows through a hydraulic coupler of inlet channel 105a that directs working fluid 105 from pump 134 to inlet 105 of expander 106.

Working fluid 104 then enters inlet 105 of expander 106 and starts expanding through multi-stage expander stator 600 and multi-stage turbine rotors of expander 106, thereby converting a portion of thermal energy of working fluid 104 into torque energy to rotate hollow shaft 107. Rotating hollow shaft 107 drives pump 134 because hollow shaft 107 is mechanically coupled to turbine rotors of pump 134. After exiting expander 106, working fluid 104 flows circumferentially and axially through discharge chamber 304 and enters recuperator heat exchanger 120 to release another portion of its thermal energy to working fluid 104 flowing through high-temperature channel 118 in opposite direction. Working fluid 104 then enters extended heat exchanger and is condensed to reset the Rankine thermodynamic cycle.

Power generator 100 shown in FIG. 5 may be configured to separate working fluid 104 at inlet 129 of pump 134 from working fluid 104 at inlet 105 of expander 106 by a seal 204. Seal 204 may sealingly surround the outer surface of hollow shaft 107. In one embodiment, seal 204 may be a non-contact seal. In another embodiment, seal 204 may be a contact seal designed to be lubricated with working fluid 104.

As best shown in FIGS. 2 and 3, hollow shaft 107 is mechanically coupled to permanent magnets 108. In one embodiment, permanent magnets 108 may be configured to provide radial load bearing surfaces for hollow shaft 107 to rotate over hydrodynamic films of working fluid 104 that wet the outer surfaces of impeller shroud 112. Hollow shaft 107 rotates concentrically with respect to impeller shroud 112 as hydrodynamic films of working fluid 104 are formed throughout annular gap 202. As hollow shaft 107 rotates and its inner surfaces are wetted by working fluid 104, hydrodynamic pressure develops within annular gap 202, effectively maintaining hollow shaft 107 levitated and concentric with respect to impeller shroud 112.

Additional radial and axial loads, exerted on hollow shaft 107 by the operations of pump 134, expander 106, and impeller 109, may be supported by tapered surfaces 203. Tapered surfaces 203 can be polished and lobed bearing surfaces extended from and mechanically coupled as part of power conversion assembly 150. For example, tapered surfaces 203 can be integral parts of hollow shaft 107. Tapered surfaces 203 can be configured to perform thrust and radial load bearing functions as working fluid 104 trapped within annular gap 202 forms hydrodynamic films between tapered surfaces 203 and correspondingly tapered portions of impeller shroud 112. In one exemplary embodiment, tapered surfaces 203 can be magnetized to perform magnetic thrust bearing functions with respect to impeller 109. In another exemplary embodiment, tapered surfaces 203 can be formed by permanent magnets oriented in a way to magnetically couple with magnetized blades 110a.

To actively control and assist stabilization of impeller 109, stator permanent magnets 305 can be configured to be part of or embedded with the structures forming shield 200. Stator permanent magnets 305 can be configured to magnetically provide a constant magnetic field and an active magnetic field through electronically controlled coils forming the stator components of stator permanent magnets 305. Electronic control of stator permanent magnets 305 can be executed through controller 212. Stator permanent magnets 305 can be further configured to produce electric power at rating sufficient to supply power to controller 212 and wireless data transmission via antenna 208 as described above with reference to FIG. 1.

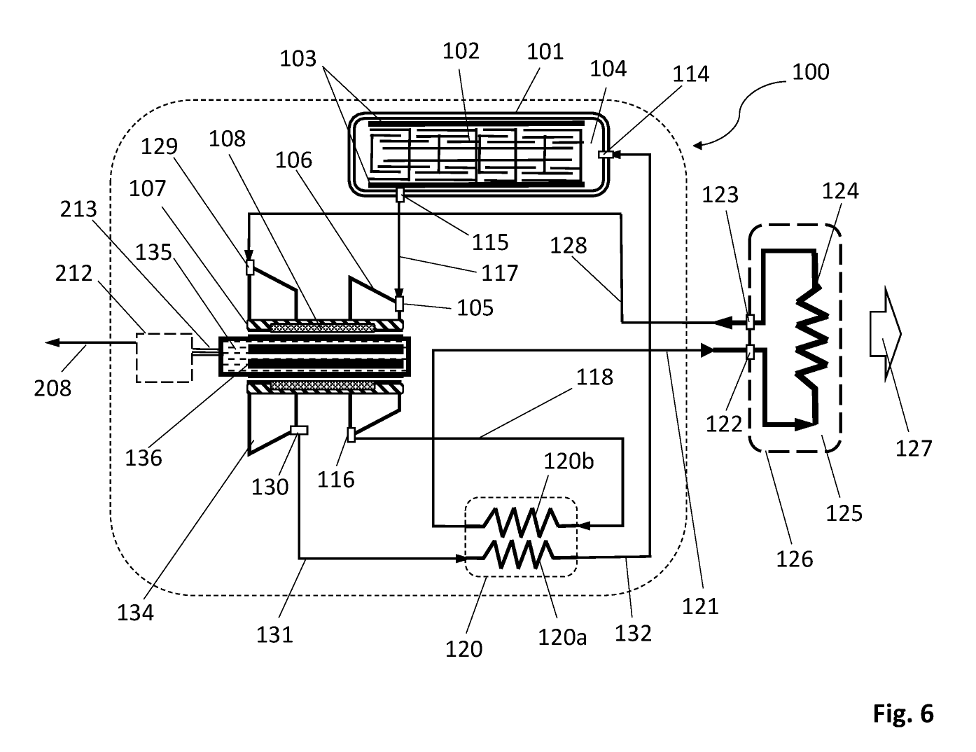

FIG. 6 is a schematic view of a power generator 100, according to another exemplary embodiment consistent with the present disclosure. Power generator 100 of FIG. 6 differs from power generators 100 and 100′ described above with reference to FIGS. 1-5 in that power generator 100 of FIG. 6 is configured to produce electricity only, whereas power generators 100 and 100′ of FIGS. 1-5 are configured to generate both electricity and torque.

More specifically, power generator 100 shown in FIG. 6 replaces impeller 109 with a magnetic stator 135 having stator poles 136. As a result, permanent magnets 108 can generate a rotary magnetic field as a result of expansion of working fluid 104 in expander 106, where the rotary magnetic field couples permanent magnets 108 with stator poles 136. Stator poles 136 may include electric coils for the purposes of converting the rotary magnetic field into electricity using a method known in the electric AC or DC generator art.

The rest of the components of power generator 100 in FIG. 6 are substantially similar to those of power generator 100 described above with reference to FIG. 1 and, therefore, the detailed descriptions of the remaining components are omitted herein.

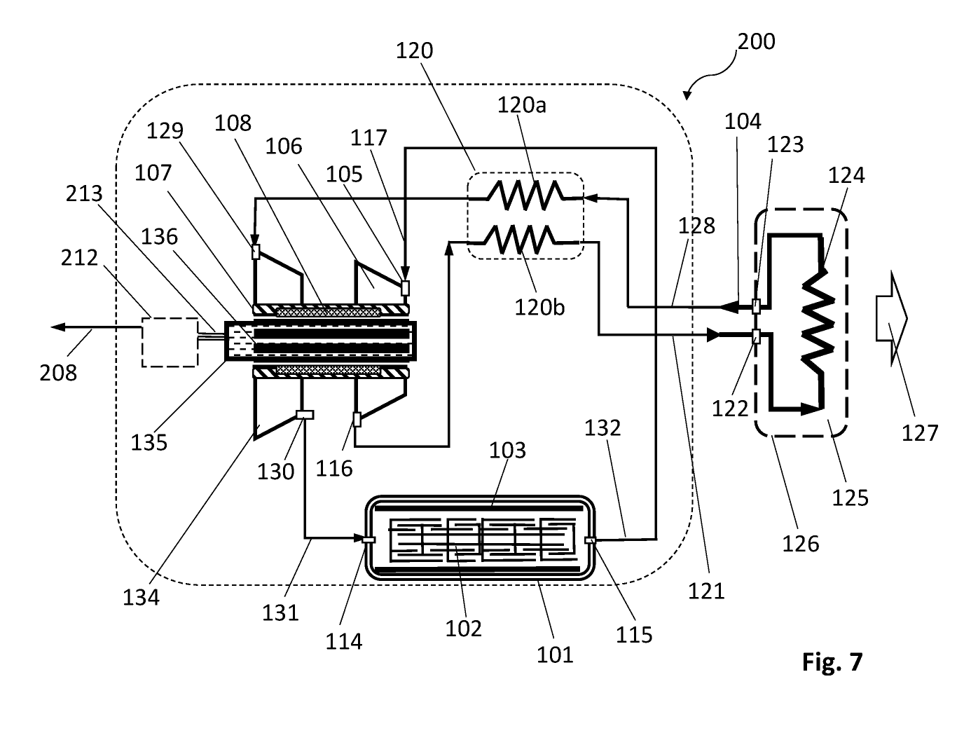

FIG. 7 is a schematic view of a power generator 100, according to another exemplary embodiment of the invention. Power generator 100 shown in FIG. 7 differs from power generator 100 shown in FIG. 6 in that recuperator heat exchanger 120 is configured to pre-heat working fluid 104 prior to entering pump 134. In this configuration, working fluid 104, with an increased energy content via thermal exchange through recuperator heat exchanger 120, is pressurized by pump 134 and flown into source heat exchanger 102 via high-temperature channel 131. After passing through source heat exchanger 102, working fluid 104 enters expander 106 via high-temperature channel 132 to expand. Like power generator 100 of FIG. 6, the rest of the components of power generator 100 of FIG. 7 are substantially similar to those of power generator 100 described above with reference to FIG. 1 and, therefore, the detailed descriptions of the remaining components are omitted herein.

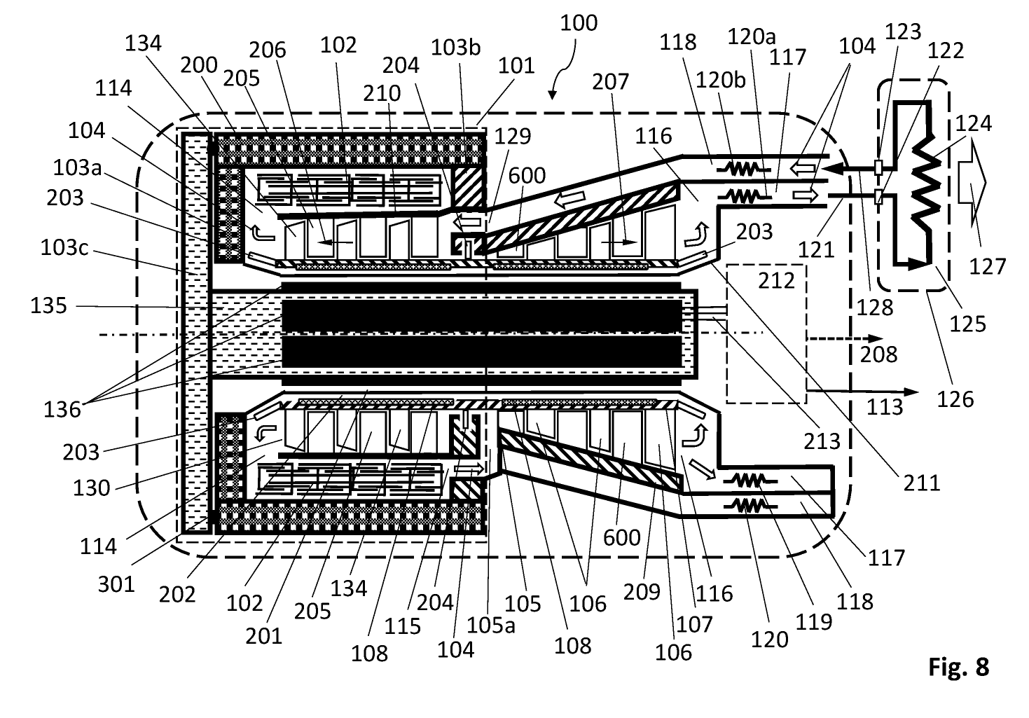

FIG. 8 is a functional, schematic diagram of a power generator 100, according to the features shown and described in FIGS. 6 and 7. FIG. 8 illustrates an exemplary configuration of power generator 100 showing in greater detail the components within housing 101 that contains, shields and thermally couples source heat exchanger 102. When source heat exchanger 102 represents thermal energy produced as a result of decaying isotopes, it can be configured to form a shielded radial thermal source embedded with heat exchanger surfaces of housing 101. In one configuration, source heat exchanger 102 can be configured to form a substantially cylindrical structure surrounding the turbo-machinery components (rotary and stationary) forming pump 134. In another configuration, source heat exchanger 102 can be configured to be further extended and surround the turbomachinery components forming expander 106.

In the exemplary embodiment shown in FIG. 8, working fluid 104 enters low-temperature channels 118 arranged to form the low- and high-temperature portions 120a and 120b of recuperator heat exchanger 120, respectively defined by substantially cylindrical thermal-hydraulic channels with heat transfer surfaces (as shown in FIG. 19) to enhance thermal energy transfer. Working fluid 104 flows from extended heat exchanger 124 into inlet 129 of pump 134 formed by one or multiple pump stators 205 arranged to be mechanically coupled to pump shroud 210.

Working fluid 104 increasingly pressurizes through the stages of pump 134 and as it pressurizes working fluid 104, it generates a pump thrust in direction 206. To mitigate or neutralize the pump thrust, the components forming expander 106 are configured to generate an expander thrust in a direction 207 opposite with respect to pump thrust direction 206. As pressurized working fluid 104 flows at the last stage of outlet expander 134, it enters source heat exchanger 102 via source inlet 114. Decay heat induced radiation is attenuated by the shields represented by the materials of source heat exchanger 102 and housing 101. In this configuration, housing 101 comprises first shield 103 and first shield front and back caps 103a and 103b.

Shield 200 further contributes to attenuating radiation. First shield front cap 103a can be configured to seal the assembly, via O-rings or other suitable seals 301, from the front portions of power generator 100. The assembly coupling to hollow shaft 107 rotates concentrically to the central portions of magnetic stator 135 by floating over hydrodynamic annular gap 202 (as shown in FIG. 5), filled by working fluid 104 forming films between the outer surface of magnetic shroud 211 and the inner surfaces (hollow portions) of hollow shaft 107. Counter opposing axial thrust and radial loads are induced by tapered surfaces 203 to ensure that hollow shaft 107 and the turbomachinery components coupled to hollow shaft 107 remain centered and concentric and maintain clearances between the stationary and rotary components. In agreement with the thermal-hydraulic schematic shown in FIG. 7, pressurized hot working fluid 104 flows out of source heat exchanger 102 and through inlet channel 105a to inlet the first stage of expander 106 through inlet 105 for expansion of working fluid 104.

As described in FIG. 5, to prevent back flow of the hot working fluid 104 back into the low-pressure channels represented by the first stages of pump 134, one or multiple seals are positioned between hollow shaft 107 and the stationary assembly mechanically coupled to the stators of pump 134 and expander 106. As for the power generator 100 configurations shown in FIGS. 1-5, hollow shaft 107 comprises rotary permanent magnets 108 configured to generate a rotary magnetic field as they are mechanically coupled to or embedded with the hollow portions of shaft 107.

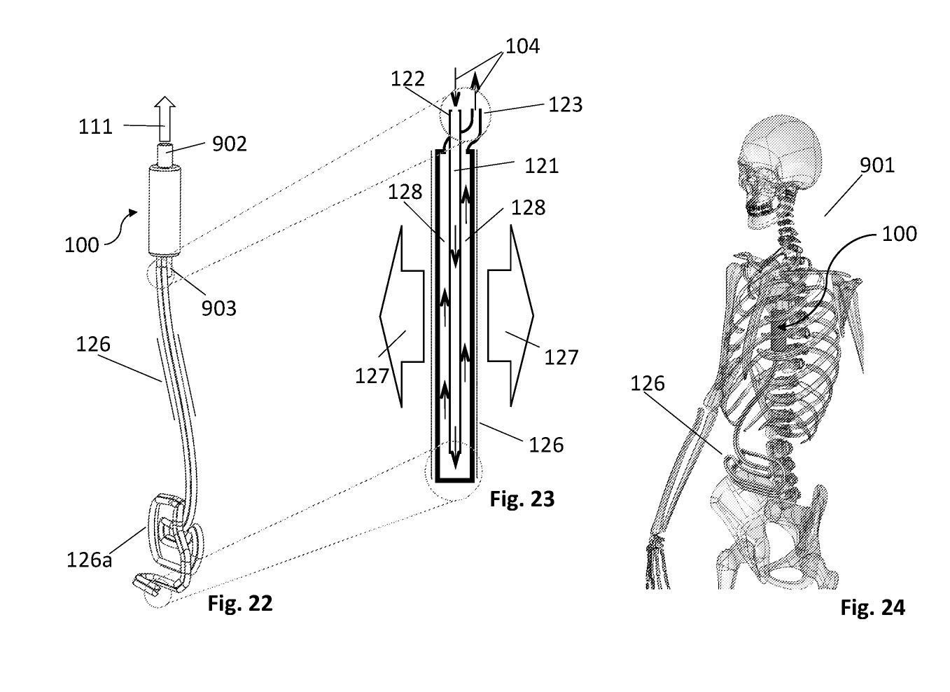

Annular gap 202 is filled with working fluid 104 to form hydrodynamic regions with pressurized working fluid 104. Supply of working fluid 104 within annular gap 202 is assisted by inlets 500 of working fluid 104 (shown in FIG. 23), where working fluid 104 is pressurized by pump 134. Pressurized working fluid 104 is also supplied to the clearance formed by tapered surfaces 203 and the outer surfaces of magnetic shroud 211. In one configuration, the first gap 201 formed by the inner surfaces of magnetic shroud 211 (hollow portions), and the outer surfaces of stator poles 136 can be configured to be filled with air or an inert gas. In another configuration, the first gap 201 formed by the inner surfaces of magnetic shroud 211 (hollow portions) and the outer surfaces of stator poles 136 can be configured to be filled with a fluid to enhance thermal transfer and cool down stator poles 136 and magnetic stator 135. As the magnetic field rotates due to the expander 106 driven rotary permanent magnets 108, the stator poles 136 magnetically couple to the rotary magnetic field and convert the magnetic energy into electricity through coils comprised by the stator poles 136.

Electricity produced by expander 106 through the magnetic stator 135 is conditioned and controlled by controller 212 so as to provide conditioned electric power outside of power generator 100 through electric line 113. In one configuration, wireless data transfer and control communications with external controllers and data acquisition can occur via antenna 208. In another configuration, data transfer and control communications with external controllers and data acquisition can occur via electric line 113 configured to carry conditioned electric power and data.

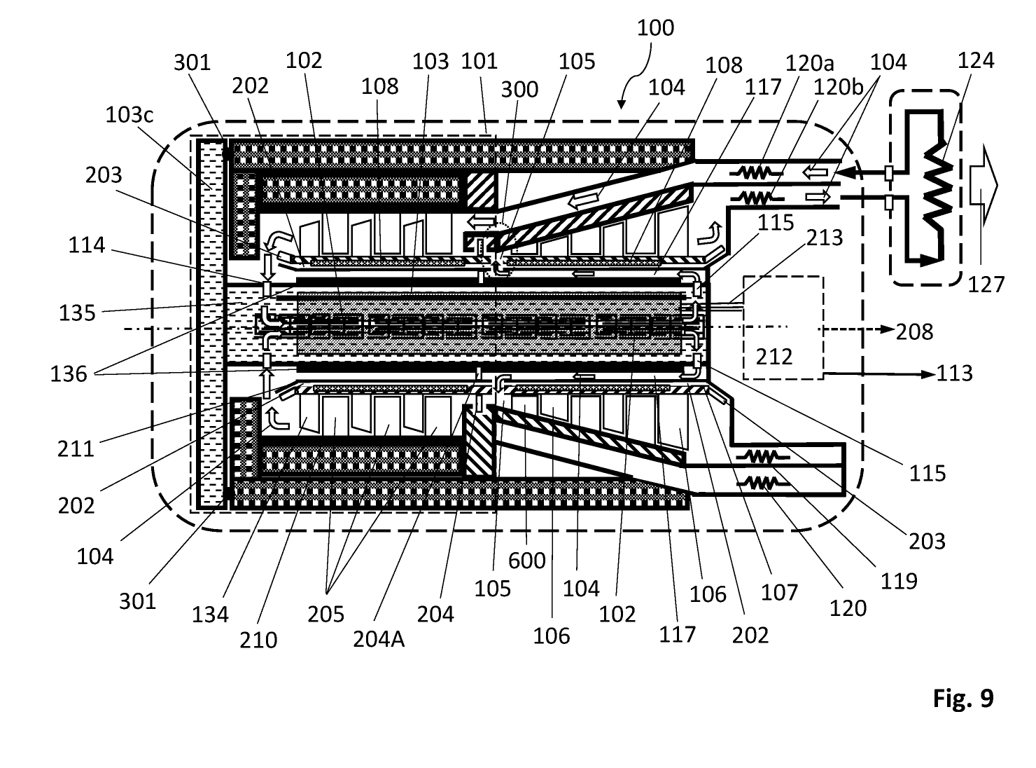

FIG. 9 is a cross-sectional view and functional schematic illustrating another exemplary embodiment of power generator 100, where source heat exchanger 102 is positioned substantially within a central location and includes the assembly forming shaft 107. In this embodiment, magnetic coupling between the rotary permanent magnets 108 and stationary stator poles 136 occurs as described in FIG. 8. In the configuration shown in FIG. 9, magnetic stator 135 comprises and shields source heat exchanger 102.

Accordingly, hollow shaft 107 is mechanically coupled to the rotary turbo-machinery components forming expander 106, pump 134 and rotary permanent magnets 108, while stationary stator poles 136 are integrated with stator 135 and source heat exchanger 102. As shown in this figure, working fluid 104 pressurized by the last stage of pump 134 enters source heat exchanger 102 through source inlet 114 (left of FIG. 9), which can be configured to allow working fluid 104 to flow across shaft 107 through a clearance or outlet formed at the edge of at least one of the tapered surfaces 203. As working fluid 104 flows through inlet 114, it enters source heat exchanger 102 forming, in this configuration, a portion of magnetic stator 135.

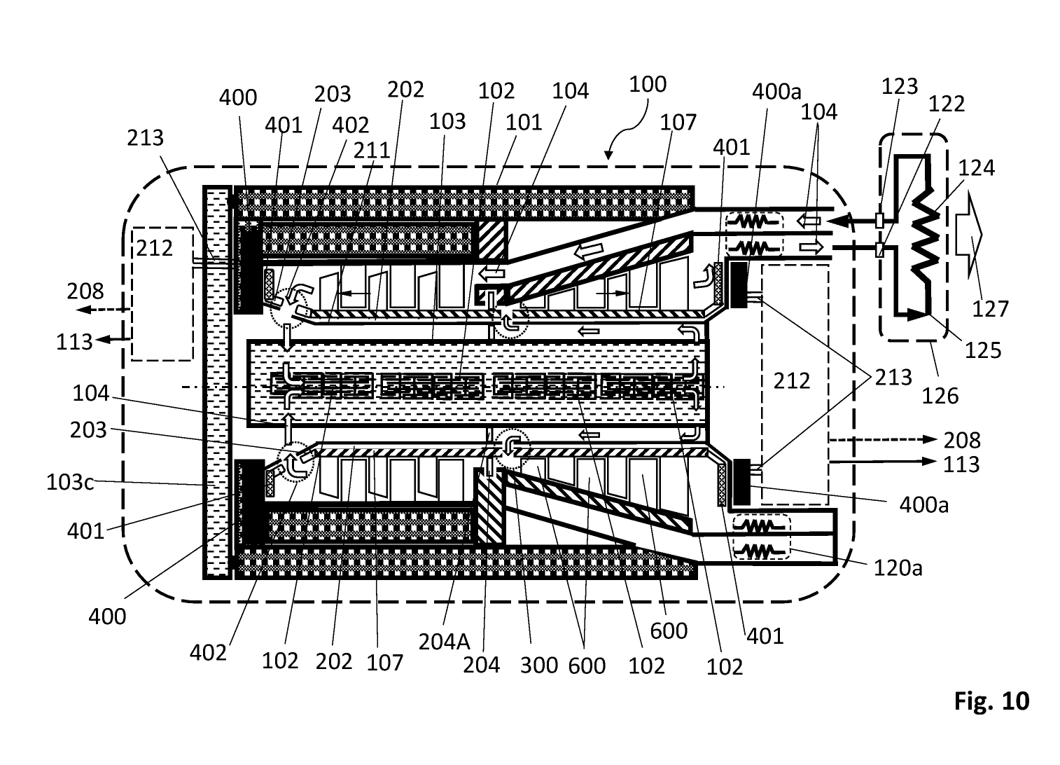

As working fluid 104 increases its energy content via thermal energy exchange with source heat exchanger 102, it flows out of source outlet 115 and enters high-temperature channel 117 formed by a substantially annular chamber comprised by the inner walls of magnetic shroud 211 and the outer walls of stator poles 136. Hot and pressurized working fluid 104 then flows into expander inlet 105 to expand through expander 106 by expanding through one or multiple expander stators 600 and proportional number of turbine rotors forming expander 106. Hot and pressurized working fluid 104 flows through rotary channels 300 (shown with more clarity in FIG. 10). As for the generator configurations described in FIGS. 5 and 8, to prevent back flow of working fluid 104 through high-temperature channels 117, first seal 204 and second seal 204A mitigate or prevent working fluid 104 leakages between the outlet of pump 134 and inlet 105 of expander 106. In this configuration, electricity produced by the coils of stator poles 136 is conditioned by controller 212 as described in FIG. 8.

FIG. 10 is a cross-sectional view and functional schematic illustrating another exemplary embodiment of power generator 100, where source heat exchanger 102 is positioned substantially within a central location as part of an assembly forming hollow shaft 107. The magnetic coupling between rotary permanent magnets and stationary electro-magnetic stators occurs through radial permanent magnets mechanically coupled to shaft 107 (hereinafter referred to as radial permanent magnets 410) and first stator 400. First stator 400 comprises electromagnetic coils and leads 213 electrically connecting to controller 212. Accordingly, radial permanent magnets 401 can be configured to be part of the thrust and radial load bearings represented by tapered surfaces 203, and bearing journal represented by magnetic shroud 211.

As for the power generator 100 described in FIG. 9, working fluid 104 executes a thermodynamic cycle as it circulates through the various components within housing 101 thermal-hydraulically coupled to extended heat exchanger 124. In this configuration, working fluid 104 enters the central portions of power generator 100 to circulate through source heat exchanger 102, crossing shaft 107 via fluid channels 402 through tapered surfaces 203 so as to also provide lubrication to these surfaces. To further control axial movement of shaft 107, radial permanent magnets 401 can be configured to provide counter-opposing magnetic forces by regulating radial first stator 400 and radial second stator 400a, both controlled by controller 212. In this configuration, radial permanent magnets 401 are coupled at both ends of shaft 107 to produce electric power by radially coupling with radial first and second stators 400 and 400a respectively.

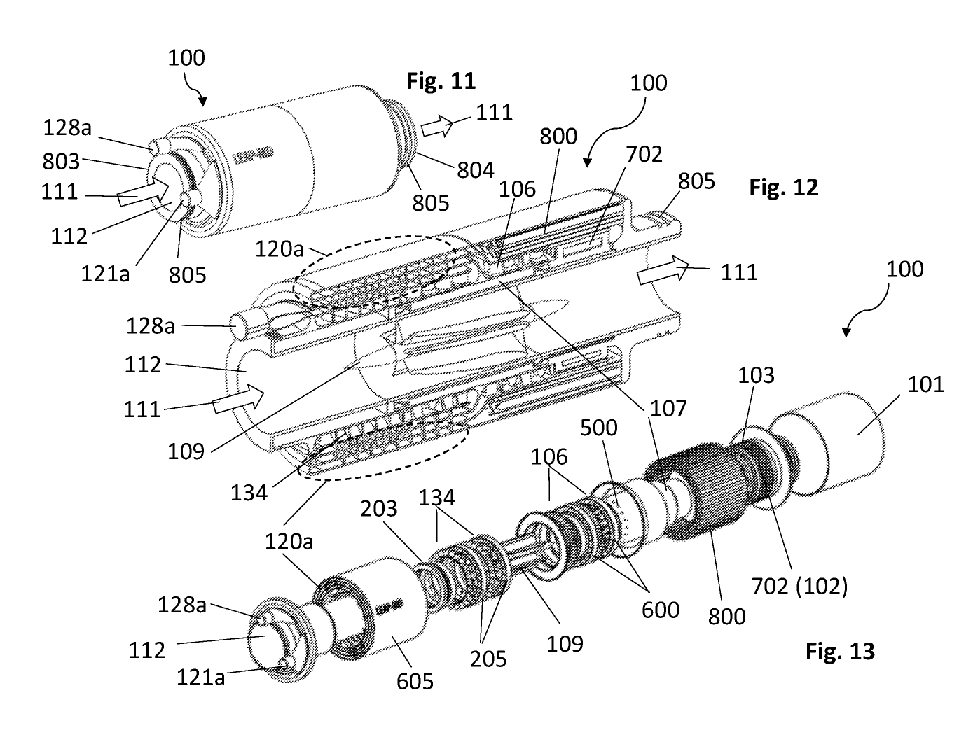

FIG. 11 illustrates an exemplary perspective view of power generator 100 described with reference to FIGS. 1-5, according to an exemplary embodiment of the invention. In this embodiment, power generator 100 is configured to convert thermal energy to pump fluid 111 by magnetically driving impeller 109. Accordingly, one end of power generator 100 is equipped with inlet 803 for fluid 111 to circulate via hydraulic channels or tubing coupled to power generator 100.

At the opposite end of power generator 100, outlet 804 provides hydraulic coupling for a hydraulic channel to enable fluid 111 to circulate out of power generator 100. Depending on the applications of power generator 100 and the physical thermal- and chemical-properties of fluid 111, inlet 803 and outlet 804 can be configured to utilize seals 805 formed by sealing materials compatible with fluid 111. When power generator 100 is configured to be implantable, for example, to support or replace LVADs or TAH applications, the hydraulic channels are represented by arteries and fluid 111 is blood. For applications employing power generator 100 as a submergible propeller, outlet 804 can be shaped as a nozzle to obtain thrust. At one end of power generator 100, working fluid 104 is configured to flow through cold inlet 128a, connected to cold channel 128 (see for example FIGS. 1-5), while hot outlet 121a provides hydraulic coupling with hot channel 121 (FIGS. 1-5). Overall, inlet and outlet 128a and 121a, respectively, provide hydraulic coupling for thermal-hydraulic channels coupled to extended heat exchanger 124 shown in FIGS. 1-10 and 24.

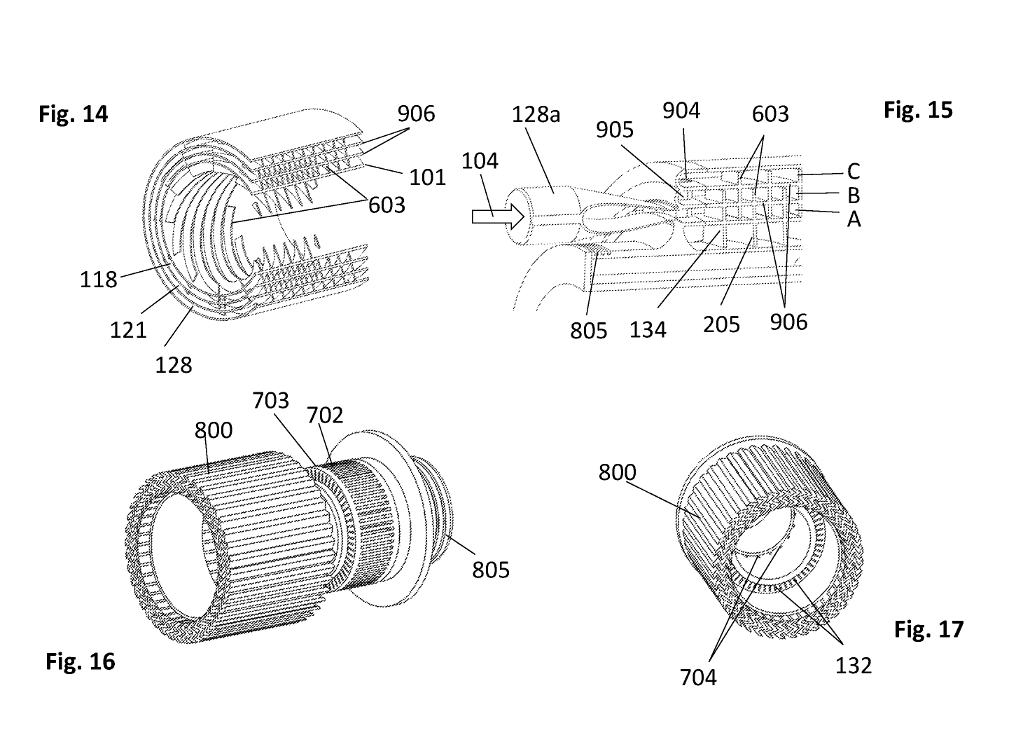

FIG. 12 is an exemplary perspective cross-sectional view of power generator 100 shown in FIG. 11, illustrating in greater details the generator internals. As also shown in FIGS. 14 and 15, recuperator heat exchanger 120 comprises multilayered channels (see the dashed area) defined by a plurality of layers 906 and a plurality of fins 603 extruding across layers 906 to provide extended heat transfer surface for working fluid 104 to exchange thermal energy when circulating through recuperator heat exchanger 120. In one configuration, layers 906 are configured to induce working fluid 104 to circulate in one direction, for example, toward the inlet of pump 134, while working fluid 104 discharged at the outlet of expander 106 and flowing in another layer 906 circulates in the opposite direction, so as to obtain a counter-flow heat exchanging mechanisms across multiple layers 906, thus enabling a higher heat exchanger effectiveness and integration within power generator 100. Therefore, working fluid 104 flows in both direction across multiple layers 906 of recuperator heat exchanger 120 throughout the circumference of power generator 100.

Given the high number of elements forming power generator 100, FIG. 12 illustrates the position of various internal components of power generator 100 with respect to one another while the exploded assembly view shown in FIG. 13 shows individual components all concentrically positioned with respect to the center line of impeller 109. To further increase the heat transfer surface areas within the power generator 100, extended recuperator 800 surrounds a rectangular and radial configuration of source 702, generically indicated as source heat exchanger 102 in FIGS. 1-10, and is configured to accommodate and shield source 702 (102).

FIG. 13 is an exemplary exploded view of power generator 100 shown in FIGS. 11 and 12, illustrating the order in which the components are assembled with respect to rotary and stationary parts of the assembly all together with recuperator heat exchanger 120, source 702 and extended recuperator 800. The configuration of the components of power generator 100 and their assembly sequence as shown in FIG. 13 reflects the schematic and functioning principles shown in FIGS. 1-5.

FIG. 14 is an exemplary perspective cross-sectional view of recuperator heat exchanger 120, showing its internal components within power generator 100 of FIG. 11 and illustrating in greater detail the extended surfaces thermally coupled across different layers 906 (see also FIG. 15) of the heat exchanger. Each layer 906 is structurally coupled to helical fins 603 to increase heat transfer surface area and working fluid 104 turbulence as it flows through annular turning channels formed by combining fins 603 with the walls forming layers 906. Each two layers 906 represent the inner and outer walls of an annular channel. Furthermore, as fins 603 extrude across multiple layers, each annular channel can be configured to represent hot- or cold-fluid channels 121, 128 and low-temperature channels 118, where working fluid 104 is cooled prior to exiting the generator and pre-heated prior to entering source heat exchanger 102 or 702, as described by the schematic and functioning diagram shown in FIGS. 1-5. Therefore, a minimum of two layers 906 define a heat transfer annular turning channel, where working fluid 104 circulates and transfers across different layers by flowing through hydraulic radial channels 904, disposed substantially radially with respect to the centerline of recuperator heat exchanger 120.

FIG. 15 illustrates a three-dimensional cut-away view of an end portion of power generator 100, showing in greater detail multiple layers 906 forming multiple annular channels A, B and C. In one configuration, working fluid 104 enters power generator 100 at inlet 128a and flows through annular channel A to transfer thermal energy with working fluid 104 circulating in counter- or parallel-flow within channels B and C. As working fluid 104 flows through the various components forming the thermodynamic cycle, it can be configured to flow back toward the portion of power generator 100 shown in this figure, and into annular channel C. This is the case, for example, in which working fluid 104 flows through extended recuperator 800, from right to left of power generator 100. Once flowing toward the end of annular channel C, working fluid 104 can cross through annular channels B and A and be hydraulically coupled to pump 134 through multiple radial channels 904. Multiple radial channel 904 are positioned throughout the circumference of recuperator heat exchanger 120 to reduce back pressure of working fluid 104 as it circulates through the internal components of power generator 100. Each radial channel can be configured to form an hydraulic passage formed by walls 905, extruding across multiple layers 906, to enable working fluid 104 circulating in one annular channel (e.g., channel A) and flow into another annular channel (e.g., channel C) without physically mixing with warmer or cooler working fluid 104 circulating in annular channel (e.g., channel B).

FIG. 16 is an exemplary partially exploded perspective view of the power generator 100 of FIG. 11, illustrating the shape of heat transfer surfaces further extending the total heat transfer surface area of the recuperator (hereinafter referred to as extended recuperator 800) with a substantially zig-zagged geometry so as to inhibit radiation from source 702 (or 102) out of source housing 703 (equivalent to housing 101 shown in FIGS. 1-5), thus executing dual functions: extending the surface areas of recuperator heat exchanger 120 to increase heat transfer with working fluid 104 and shielding radiation potentially emitted by source 702 (equivalent to source heat exchanger 102 in FIGS. 1-5).

FIG. 17 is an exemplary perspective view with a different angle of the extended recuperator 800 of power generator 100 shown in FIG. 11, illustrating high-temperature channels 132. In this configuration, the heat source (e.g., alpha emitting source) is embedded with the source housing 703 (FIG. 16), and working fluid 104 is pressurized through high-temperature channels 132 through radial inlet/outlet channels 704 to execute energy exchange between source 702 and working fluid 104 circulating through source housing 703.

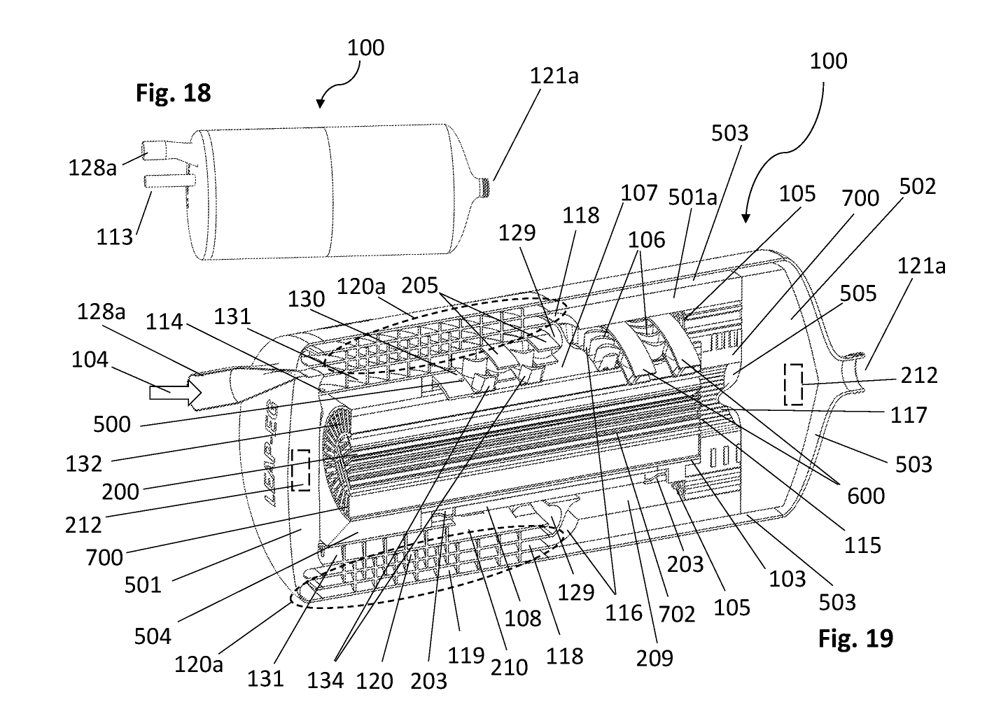

FIG. 18 is a perspective view of power generator 100 described with reference to FIGS. 6-10, illustrating power generator 100 configured to convert thermal energy into electricity. Working fluid 104 enters power generator 100 at the cold inlet 128a and exits at hot outlet 121a. Depending on applications, cold inlet 128a and hot outlet 121a can be reversed (e.g., working fluid 104 flowing hot out of outlet 128a and cold into inlet 121a), and power generator 100 converts thermal energy into conditioned electricity distributed by electric line 113.

FIG. 19 is a perspective cross-sectional view of power generator 100 described above with reference to FIG. 18, illustrating the generator internal components configured to substantially surround and shield the thermal source. Power generator 100 shown in this figure is configured to solely produce electricity, however the rotary and stationary turbomachinery components described for power generator 100 shown in FIGS. 11-19 are substantially similar.

As shown in FIG. 19, hydraulic channels 500 are more clearly visible. In one exemplary configuration of power generator 100, hydraulic channels 500 represent a series of radially distributed flow channels on hollow shaft 107 assembly (also generically shown in the schematic of FIG. 10 under fluid channels 402 and rotary channels 300). Hydraulic channels 500 enable working fluid 104 to flow across hollow shaft 107 to supply working fluid 104 to tapered surfaces 203 or provide flow paths for working fluid 104 to inlet/outlet stationary source assembly 700.

Additionally, the multi-stage rotary components of pump 134 and expander 106 are shown along with multi-stage pump stators 205 and expander stator 600. As source 702 (equivalent to 102) is positioned concentrically, substantially in the central portions of power generator 100, inside source assembly 700, working fluid 104 flows through the high-temperature channel 132 (source heat exchanger and shield) through hydraulic channels 500. More generally, working fluid 104 flows through the various components forming power generator 100 to execute energy exchange starting with recuperator heat exchanger 120 (shown within dashed areas). Working fluid 104 is then pressurized by pump 134 prior to entering source assembly 700, where working fluid 104 increases its energy content. Working fluid 104 flows through source assembly 700 and expands through rotary components of expander 106 to convert the energy of working fluid 104 into mechanical energy in the form of torque at shaft 107.

Sets of rotary permanent magnets 108 (or 401 for power generator 100 configured as shown in FIG. 10) are mechanically coupled to shaft 107 to generate a rotary magnetic field, further coupled to axial or radial electro-magnetic coils (not shown in this figure but designated with reference number 136 in FIG. 8, and reference number 400 in FIG. 10) to produce electricity.

The electricity produced by thermal conversion of working fluid 104 into electric power is controlled and conditioned by controller 212, shown embedded with thermal and radiation shield 502 and/or embedded with shield 501. As working fluid 104 discharges at outlet 116 of expander 106, it enters the central annular channel of recuperator heat exchanger 120 to transfer thermal energy to working fluid 104 that is flowing in counter-flow configuration and is thermally coupled by the annular channels comprised by recuperator heat exchanger 120. In some configurations, working fluid 104 further circulates through internal flow pathways (not shown) into the extended heat exchanger comprised by source assembly 700. As working fluid 104 flows toward the hot outlet 121a of power generator 100, it provides thermal and radiation shield through a jacket 503 configured to substantially surround radial shield 501a, wherein radial shield 501a comprises the expander shroud 209.

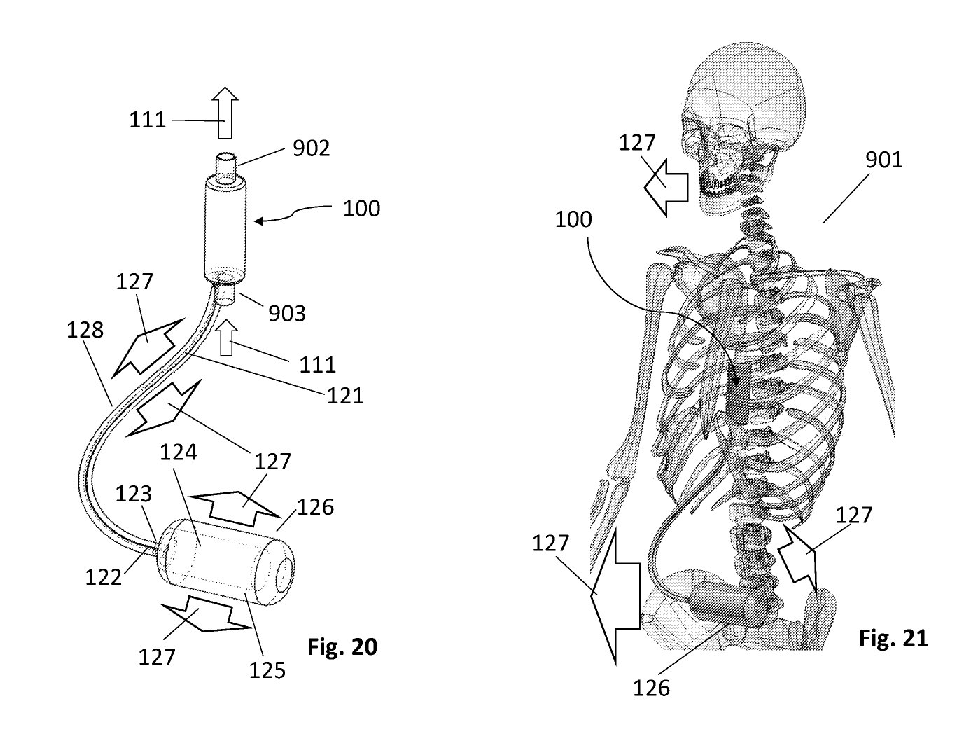

FIG. 20 is a perspective view of power generator 100 described in FIGS. 8-19 and configured as shown in FIG. 11, which is coupled to extended heat exchanger 124 by hot and cold channels 121 and 128, respectively, for use in a LVAD or TAH, according to an exemplary embodiment of the present disclosure. Hot and cold channels 128 and 121 are configured to extend the heat transfer surfaces from recuperator heat exchanger 120, comprised by power generator 100 housing, to further extended heat transfer surfaces wetted by working fluid 104 as it flows through these hot and cold thermal-hydraulic channels coupling power generator 100 to the extended heat exchanger 124. In this configuration, hot and cold channels 121 and 128 form a heat exchanger thermally coupled with the ultimate heat sink 127 through the patient body 901, shown in FIG. 21 and represented by tissues, body fluids, bones, skin, inhaled and exhaled air, sweat, etc.

FIG. 21 is a transparent perspective view of the power generator 100 and extended heat exchanger 124 of FIG. 20, illustrating the approximate position of power generator 100 and extended heat exchanger 124 when implanted in a patient body 901. In this configuration, fluid 111 is blood flowing from/to arteries or from/to heart ventricles in/out of power generator 100 via LVAD hydraulic coupling 903 (e.g., aorta) and 902 (e.g., ventricle). Hot and cold channels 121 and 128 and extended heat exchanger 124 are thermally coupled with body 901 internals to transfer thermal energy rejected by the closed-loop Rankine cycle actuated by power generator 100. In this configuration, thermal energy rejected by the Rankine cycle is mainly transferred from the extend heat exchanger 124 to the body 901 internals via second thermal interface 126.

FIG. 22 is a perspective view of power generator 100 of FIG. 11, coupled to a variation of extended heat exchanger 124 as the heat transfer surfaces characterizing hot and cold channels 121 and 128 are further extended to define the entirety of extended heat exchanger 124 heat transfer surfaces, according to another exemplary embodiment of the present disclosure. In this configuration, the length of hot and cold channels 121 and 124 can be configured to be extended to further increase the surface area exposed to body 901 internal tissues, fluids, bones etc., to further rejecting thermal energy discharged by the Rankine cycle to the ultimate heat sink 127 (e.g. air surrounding body 901). In this configuration, hot and cold channels 121 and 128 further distribute temperature through body 901 as working fluid 104 condenses through thermal transfer with the body 901 and the ultimate heat sink 127. The extended hot and cold channels 121 and 128 can be configured to be comprised by the second thermal interface 126 described in FIG. 1.

FIG. 23 is a functional schematic diagram of power generator 100 and extended heat exchanger 126a of FIG. 22, illustrating the flow patterns of working fluid 104 as working fluid circulates in and out of power generator 100 and through the hot and cold channels 121 and 128, respectively. Hot working fluid 104 discharged by expander 106 and exiting power generator 100 after energy exchange with recuperator heat exchanger 120 flows internally through a flexible heat exchanger 126a comprising hot and cold channels 121 and 128, respectively, and second thermal interface 126 so as to enable positioning within body 901 as shown in FIG. 24. To protect body 901 internals from the highest temperature represented by working fluid 104 as it cools down through energy exchange with body 901, the hot channel 121 is positioned substantially centrally with respect to cold channel 128, where cold channel 121 can be configured to substantially surround hot channel 121.

FIG. 24 is a transparent perspective view of power generator 100 and extended heat exchanger 126a of FIGS. 22 and 23, according to another exemplary embodiment of the present disclosure. In this illustration, the approximate positions of power generator 100 is shown along with flexible extended heat exchanger 126a which can be configured for positioning in, for example, the abdominal regions of body 901 to enhance energy exchange with body 901 while minimizing hot temperature spots as working fluid 104 cools down while flowing throughout the flexible heat exchanger.

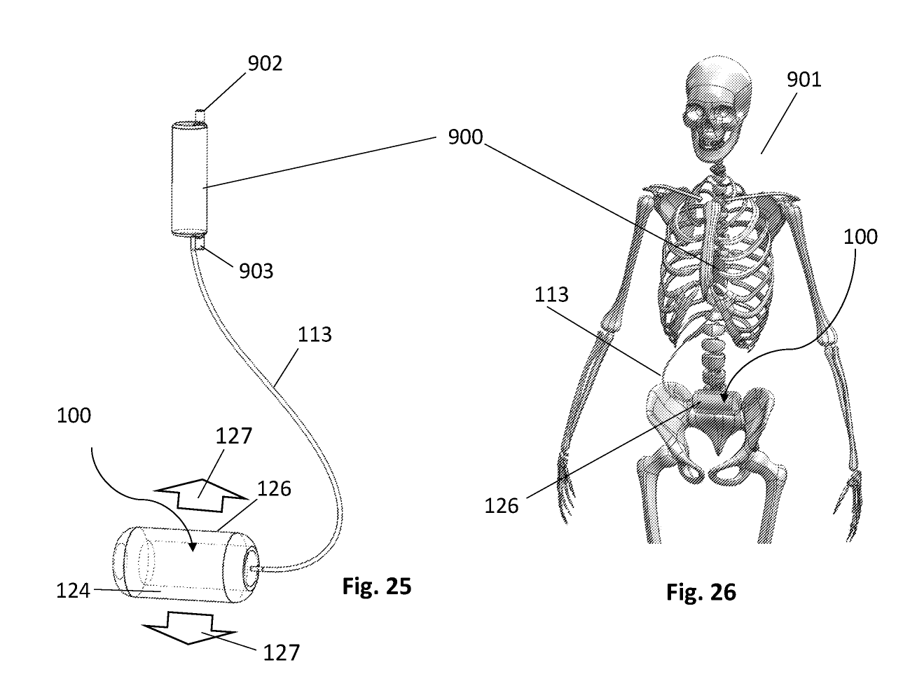

FIG. 25 illustrates an application of power generator 100 when configured to supply electric power via electric line 113 to a FDA-approved LVAD 900. In this configuration, power generator 100 may include extended heat exchanger 124 and/or flexible heat exchanger 126a shown in FIGS. 22-24. This configuration of power generation 100 is described with reference to FIGS. 6-10, 18, and 19. In this configuration, power generator 100 converts thermal energy from source heat exchanger 102 or 702 into conditioned electricity, distributed outside of power generator 100 by electric line 113.

FIG. 26 is a transparent perspective view of power generator 100 and extended heat exchanger 124 of FIG. 25, illustrating exemplary positions of power generator 100 and extended heat exchanger 124 when implanted in a patient body. Power generator 100 comprises all the components described, for example, in FIGS. 18, 20, 22, and 23 so as to provide an electric generator fully encapsulated within the second thermal interface 126.

For all non-implantable applications (e.g., robotics), power generator 100 can be configured to include the heat exchangers configured to transferring thermal energy to the ultimate heat sink 127, namely, extended heat exchanger 124, flexible heat exchanger 126a and the heat exchanger represented by the hot and cold channels 121 and 128, respectively. Alternatively, depending on the application, for non-implantable applications, power generator 100 can be positioned at a distance from the extended heat exchanger 124, which can be represented by a finned radiator configured to condense working fluid 104.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A medical device for displacing a bodily fluid inside a patient's body, the device comprising:
a source heat exchanger containing a heat generating source and being configured to transfer heat from the heat generating source to a working fluid;
a hollow shaft comprising a plurality of permanent magnets;
an impeller shroud disposed inside the hollow shaft, the impeller shroud defining an internal passageway through which the bodily fluid passes through;
an impeller disposed inside the internal passageway of the impeller shroud, the impeller being magnetically coupled to the permanent magnets of the hollow shaft; and
an expander comprising a rotary component mechanically coupled to the hollow shaft, the expander being driven by the working fluid flowing from the source heat exchanger to rotate the hollow shaft,
wherein rotation of the hollow shaft generates a rotary magnetic field in the hollow shaft to cause the impeller to rotate and displace the bodily fluid flowing through the internal passageway.

2. The medical device of claim 1, wherein the impeller shroud is stationary.

3. The medical device of claim 1, wherein the impeller shroud is concentrically disposed within the hollow shaft with respect to a rotational axis of the impeller.

4. The medical device of claim 1, wherein the bodily fluid is blood, and the internal passageway is in fluid communication with a portion of the heart of the patient.

5. The medical device of claim 1, further comprising a pump comprising a rotary component mechanically coupled to the hollow shaft.

6. The medical device of claim 1, wherein the medical device is configured to be implanted inside the patient's body.

7. The medical device of claim 1, wherein the heat generating source comprises a nuclear isotope emitting alpha-particles.

8. The medical device of claim 7, wherein the nuclear isotope comprises Plutonium-238.

9. The medical device of claim 1, further comprising a radial magnet coupled to the hollow shaft to generate a variable magnetic field when the hollow shaft rotates.

10. The medical device of claim 9, further comprising an electromagnetic stator configured to convert the variable magnetic field into electricity.

11. A power generator for a medical device comprising:
a source heat exchanger containing a heat generating source and being configured to transfer heat from the heat generating source to a working fluid;
a hollow shaft comprising a plurality of permanent magnets;
an expander comprising a rotary component mechanically coupled to the hollow shaft, the expander being driven by the working fluid flowing from the source heat exchanger to rotate the hollow shaft; and
a magnetic stator disposed inside the hollow shaft, the magnetic stator and comprising a plurality of stator poles magnetically coupled to the plurality of permanent magnets of the hollow shaft,
wherein rotation of the hollow shaft generates a rotary magnetic field in the hollow shaft, and the plurality of stator poles are configured to convert the rotary magnetic field into electricity.

12. The power generator of claim 11, wherein the medical device is configured to be implanted inside a patient's body for displacing a bodily fluid inside the patient's body.

13. The power generator of claim 12, wherein the power generator is disposed outside the patient's body and is connected to the medical device through a power line.

14. The power generator of claim 11, further comprising a controller configured to receive the electricity from the plurality of stator poles and to condition the electricity to the medical device.

15. The power generator of claim 11, wherein the source heat exchanger is disposed inside the hollow shaft.

16. The power generator of claim 15, wherein the magnetic stator is integrated with the source heat exchanger.

17. The power generator of claim 11, wherein the magnetic stator is stationary.

18. The power generator of claim 11, further comprising a pump comprising a rotary component mechanically coupled to the hollow shaft.

19. The power generator of claim 11, wherein the heat generating source comprises a nuclear isotope emitting alpha-particles.

20. The power generator of claim 19, wherein the nuclear isotope comprises Plutonium-238.
